(19) United States
(12) Patent Application Publication
(10) Pub. No.: US 2021/0401870 A1
(43) Pub. Date: Dec. 30, 2021

(54) USE OF SOY HULL POLYSACCHARIDE IN PREPARATION OF DRUGS FOR MODULATING INTESTINAL FLORA, REDUCING BLOOD GLUCOSE AND BLOOD LIPID, AND RESISTING INFLAMMATION, AND OF FOODS FOR SPECIAL MEDICAL PURPOSE

(71) Applicant: Bohai University, Jinzhou City (CN)

(72) Inventors: He LIU, Jinzhou City (CN); Lina YANG, Jinzhou City (CN); Qian LIN, Jinzhou City (CN); Danshi ZHU, Jinzhou City (CN); Jun LIU, Jinzhou City (CN); Chunfang MA, Jinzhou City (CN); Jinghang HUANG, Jinzhou City (CN); Yafan ZHAO, Jinzhou City (CN); Shunxiu LI, Jinzhou City (CN); Hong SONG, Jinzhou City (CN); Shengnan WANG, Jinzhou City (CN); Lijie ZHU, Jinzhou City (CN); Jun LI, Jinzhou City (CN); Yutang HE, Jinzhou City (CN)

(21) Appl. No.: 17/003,714

(22) Filed: Aug. 26, 2020

(30) Foreign Application Priority Data
Jun. 24, 2020 (CN) 202010591008.5

(51) Int. Cl. A61K 31/715; A61K 36/185

(52) U.S. Cl. CPC A61K 31/715; A61K 36/185; A61K 2236/37; A61K 2236/17; A61K 2236/15

(57) ABSTRACT

The present invention provides use of a soy hull polysaccharide in the preparation of drugs for modulating intestinal flora, reducing blood glucose and blood lipid, and resisting inflammation, and of foods for special medical purpose (FSMPs), and the present invention belongs to the technical field of use of a soy hull polysaccharide. The soy hull polysaccharide provided in the present invention, with an interfacial activity in the intestinal tract, can slow down the absorption of cholesterol or the like; and has the activities for modulating intestinal flora, reducing blood glucose and blood lipid, and preventing intestinal inflammation. The soy hull polysaccharide can be used for preparing drugs for modulating intestinal flora, reducing blood glucose and blood lipid, and preventing and/or treating intestinal inflammation, and for preparing FSMPs.

### TECHNICAL FIELD

The present invention belongs to the technical field of use of a soy hull polysaccharide, and in particular to the use of a soy hull polysaccharide in the preparation of drugs for modulating intestinal flora, reducing blood glucose and blood lipid, and resisting inflammation, and of foods for special medical purpose (FSMPs).

### BACKGROUND

A soy hull polysaccharide, a biological macromolecule prepared from soy hulls, is a hydrocolloid with thickening, gelatinizing and emulsifying properties, which can be used as a natural food emulsifier. At present, the research on the soy hull polysaccharide mainly focuses on the extraction methods, emulsification, gelatinization and other use, but there is rare research on the structure and functional activities of the soy hull polysaccharide.

Especially, the functional research on the soy hull polysaccharide mostly focuses on the application of emulsifying properties at present, but the use of the soy hull polysaccharide in other aspects has not been reported.

### SUMMARY

In view of this, the present invention is intended to provide the use of a soy hull polysaccharide in the preparation of drugs for modulating intestinal flora, reducing blood glucose and blood lipid, and resisting inflammation, and in the preparation of FSMPs. The soy hull polysaccharide provided in the present invention, with an interfacial activity in the intestinal tract, can slow down the absorption of cholesterol or the like; and has the activities for reducing blood glucose and blood lipid and preventing intestinal inflammation.

In order to realize the objective of the present invention, the present invention provides the following technical solutions.

The present invention provides use of a soy hull polysaccharide in the preparation of drugs for modulating intestinal flora and FSMPs.

Preferably, the modulation includes promoting the proliferation of probiotics and increasing the diversity of intestinal flora.

The present invention also provides the use of a soy hull polysaccharide in the preparation of drugs for reducing blood glucose and blood lipid, and in the preparation of FSMPs.

Preferably, the hyperglycemia includes type II diabetes.

The present invention also provides the use of a soy hull polysaccharide in the preparation of drugs for preventing and/or treating inflammation, and in the preparation of FSMPs.

Preferably, the inflammation includes colitis.

Preferably, a preparation method of the soy hull polysaccharide includes the following steps:

1) crushing soy hulls and mixing the crushed soy hulls with an ethanol solution; stirring the resulting mixture at 15° C. to 25° C. for 25 min to 35 min; and then conducting filtering and drying to obtain a coarse material of soy hulls; where the ethanol solution has a volume fraction of 0.8% to 1.2%, and the soy hulls have a mass-volume ratio of 1 g:(8-12) mL with the ethanol solution; and

2) mixing the soybean seed coat coarse material with an extractant solution; conducting microwave-assisted extraction (MAE) and then liquid-solid separation for the resulting mixture; and subjecting the obtained liquid phase to concentration and alcohol precipitation to obtain a precipitate, namely, a soy hull polysaccharide;

where, the MAE is conducted at 83° C. to 88° C. for 30 min to 40 min, with power of 450 W to 500 W;

the soybean seed coat coarse material has a mass-volume ratio of 1 g:(15-25) mL with the extractant solution, and the extractant solution has a mass fraction of 0.5% to 0.7%; and

the extractant solution includes an ammonium oxalate solution.

Preferably, in step 2), after the concentration, the method further includes adjusting the pH of the concentrated solution to 3.9 to 4.1; and absolute ethanol is used in the alcohol precipitation at a volume that is 2.8 to 3.2 times the volume of the concentrated solution.

Preferably, the soy hull polysaccharide has a structure shown in formula I:

formula I, where, ● is GalA, [symbol] is Ara, ▴ is Rha, and ▪ is Gal.

Beneficial effects of the present invention: the present invention provides the use of a soy hull polysaccharide in the preparation of drugs for modulating intestinal flora, reducing blood glucose and blood lipid, and preventing inflammation, and in the preparation of FSMPs. The soy hull polysaccharide provided in the present invention, with an interfacial activity in the intestinal tract, can slow down the absorption of cholesterol or the like; and has the activities for reducing blood glucose and blood lipid, and preventing and/or treating intestinal inflammation.

The soy hull polysaccharide provided by the present invention plays a role in modulating the intestinal flora, and can significantly promote the proliferation of probiotics. The soy hull polysaccharide provided by the present invention has a stimulating effect on the growth of fecal bacteria that is more significant than inulin (which is considered to be an indigestible food ingredient that selectively stimulates the growth of probiotics in the colon). The soy hull polysaccharide provided by the present invention can also increase the diversity of intestinal flora, and enrich the species of intestinal bacteria.

The soy hull polysaccharide provided by the present invention can reduce the fasting blood glucose (FBG) concentration. The soy hull polysaccharide, in combination with the mucus barrier with high viscosity and hydrogel properties, inhibits the rapid absorption of lipid and glucose, resulting in the reduction of FBG and triglyceride (TG). Therefore, the soy hull polysaccharide has the efficacy of reducing blood glucose and blood lipid, and can prevent the occurrence of diabetes.

The soy hull polysaccharide provided by the present invention can improve the symptoms of mice with colitis, including body weight loss, diarrhea and intestinal bleeding. The soy hull polysaccharide provided by the present invention has strong hydrability, and can increase the stool weight, slow down the absorption of nutrients in the intestine, and alleviate the diarrhea and intestinal bleeding.

The present invention provides the use of a soy hull polysaccharide in the preparation of FSMPs, and the soy hull polysaccharide serves as a functional factor in the preparation of FSMPs. The soy hull polysaccharide can be consumed for a long time without toxic and side effects, and is beneficial to human health.

### BRIEF DESCRIPTION OF DRAWINGS

FIG. 1 shows the influence of the soy hull polysaccharide on the body weight, water and food intake, and serum FBG, TG and HDL-c levels of rats with a high-fat and high-sugar diet.

FIG. 2 shows the influence of the soy hull polysaccharide on the intestinal flora of rats with a high-fat and high-sugar diet, where, (a) shows the influence at the phylum-level; (b) shows the influence at the genus-level, (c) shows the principal component analysis (PCA) for the structure of the intestinal flora; and (d) shows a heat map of PCA for the intestinal flora based on weighted unifrac.

FIG. 3 is a heat map for the clustering, functions and abundance of the intestinal flora in rats treated with the soy hull polysaccharide.

FIG. 4 shows the influence of the soy hull polysaccharide on the occurrence of type II diabetes in rats.

FIG. 5 shows the influence of the soy hull polysaccharide on BALB/C mice with DSS-induced colitis, where, (A) shows the change in the body weight during treatment; (B) shows the body weight gain during treatment; (C) shows the diarrhea index; and (D) shows the bleeding index.

FIG. 6 shows the influence of the soy hull polysaccharide on the TNF-α, intestinal tissue morphology and TLR-4/NF-κB pathway in mice with DSS-induced colitis, where, (A) shows the ELISA analysis for TNF-α in the serum of mice with colitis; (B) shows the hematoxylin and eosin (HE) staining for colon tissues; (C) shows the western blotting analysis for the relative expression of TLR4 and NF-κB in colon tissues; and (D) shows the gray analysis for the relative expression of TLR4 and NF-κB.

FIG. 7 shows the response of the intestinal floras in all groups of Example 3 to the treatment of the soy hull polysaccharide, where, (A) shows the PCA score for the intestinal flora; (B) shows the 3D-PCoA for the intestinal flora; (C) shows the relative abundance for the intestinal flora at the phylum-level; and (D) shows the relative abundance for the intestinal flora at the genus-level.

### DETAILED DESCRIPTION

The present invention provides the use of a soy hull polysaccharide in the preparation of drugs for modulating intestinal flora and FSMPs. In the present invention, the soy hull polysaccharide is prepared preferably by MAE, with soy hulls as a raw material, and oxalic acid or ammonium oxalate as an extractant. In a preferred embodiment of the present invention, the soy hull polysaccharide has a structure shown in formula I:

formula I, where, ● is GalA, [symbol] is Ara, ▴ is Rha, and ▪ is Gal.

In the present invention, the soy hull polysaccharide can promote the proliferation of intestinal probiotics, and can increase the diversity of intestinal flora. The present invention has no special limitation on the dosage forms of drugs for modulating intestinal flora and FSMPs, and conventional dosage forms in the art may be adopted, including but not limited to powder, tablet, capsule, pill, granule, and liquid oral preparation. In the present invention, the drugs for modulating intestinal flora and FSMPs also include an adjuvant. The present invention has no special limitation on the type and dosage of the adjuvant, and a conventional type and dosage of the adjuvant in the art may be adopted.

The present invention also provides the use of a soy hull polysaccharide in the preparation of drugs for reducing blood glucose and blood lipid, and in the preparation of FSMPs. In the present invention, the soy hull polysaccharide has influence on the body weight, water and food intake, and serum FBG, TG and HDL-c levels of a rat with a high-fat and high-sugar diet, which can reduce the FBG concentration and prevent the occurrence of diabetes. In the present invention, the soy hull polysaccharide reduces the blood glucose and blood lipid based on principles including the following aspects: (1) The soy hull polysaccharide, with prominent viscosity and gelatinization, can delay the diffusion of glucose and reduce the FBG level. (2) The soy hull polysaccharide, in combination with the mucus barrier with high viscosity and hydrogel properties, inhibits the rapid absorption of lipids and glucose, resulting in the reduction of FBG and TG. (3) The soy hull polysaccharide can significantly increase the serum high-density lipoprotein cholesterol (HDL-C) content. (4) The soy hull polysaccharide can improve the intestinal flora imbalance caused by a high-fat and high-sugar diet without dietary fiber, achieving the reduction of the blood glucose and blood lipid by modulating the intestinal flora.

The present invention has no special limitation on the dosage forms of drugs for reducing blood glucose and blood lipid and FSMPs, and conventional dosage forms in the art may be adopted, including but not limited to powder, tablet, capsule, pill, granule, and liquid oral preparation. In the present invention, the drugs for reducing blood glucose and blood lipid and FSMPs also include an adjuvant. The present invention has no special limitation on the type and dosage of the adjuvant, and a conventional type and dosage of the adjuvant in the art may be adopted.

The present invention also provides the use of a soy hull polysaccharide in the preparation of drugs for preventing and/or treating inflammation, and in the preparation of FSMPs. In the present invention, the inflammation preferably includes colitis. In the present invention, the soy hull polysaccharide improves the colitis in mice by modulating the intestinal flora. The present invention has no special limitation on the dosage forms of drugs for preventing and/or treating inflammation and FSMPs, and conventional dosage forms in the art may be adopted, including but not limited to powder, tablet, capsule, pill, granule, and liquid oral preparation. In the present invention, the drugs for preventing and/or treating inflammation and FSMPs also include an adjuvant. The present invention has no special limitation on the type and dosage of the adjuvant, and a conventional type and dosage of the adjuvant in the art may be adopted.

In the present invention, the soy hull polysaccharide is preferably the only active ingredient in the drugs and FSMPs, and the soy hull polysaccharide has a mass percentage preferably of 0.1% to 99% in the drugs and FSMPs.

In the present invention, a preparation method of the soy hull polysaccharide includes the following steps:

1) soy hulls are mixed with an ethanol solution, and the resulting mixture is stirred at 15° C. to 25° C. for 25 min to 35 min, then filtered, and dried to obtain a coarse material of soy hulls; and

2) the coarse material of soy hulls is mixed with an extractant solution, and subjected to MAE and then liquid-solid separation; and the obtained liquid phase is subjected to concentration and alcohol precipitation to obtain a precipitate, namely, a soy hull polysaccharide.

In the present invention, the soy hulls are preferably dried and crushed soy hulls. The present invention has no special limitation on the drying method for the soy hulls, and a conventional drying method in the art may be adopted. In the present invention, the soy hulls, after crushed, are preferably sieved preferably by a 20-mesh sieve, so as to remove protein residues and impurities. In the present invention, the soy hulls, after sieved by a 20-mesh sieve, are preferably crushed once again preferably by Twister, and then preferably sieved by a 60-mesh sieve; and the sieved components are collected. In the present invention, the ethanol solution has a volume fraction preferably of 0.8% to 1.2%, and more preferably of 1%; and the soy hulls have a mass-volume ratio, preferably of 1 g:(8-12) mL and more preferably of 1 g:10 mL, with the ethanol solution. In the present invention, the mass-volume ratio of the soy hulls to the ethanol solution is not limited to the g:mL level, and it can be scaled up or down in the same proportion, such as kg:L. In the present invention, the soy hulls are mixed with the ethanol solution to allow impurities such as lipids to be dissolved, thereby promoting the precipitation of polysaccharides. In the present invention, the mixture has a temperature preferably of 20° C., and the stirring is conducted preferably for 30 min. In the present invention, the filtering is conducted preferably with double-layer gauze, and the filter cake obtained from the filtering is collected and dried to obtain a coarse material of soy hulls. In the present invention, the drying is preferably oven drying, and the oven drying is conducted preferably at 63° C. to 68° C., and more preferably at 65° C. The present invention has no special limitation on the time of the oven drying, provided that the material is dried to constant weight. The drying equipment is preferably a blast drying oven.

In the present invention, the coarse material of soy hulls is mixed with an extractant solution, and subjected to MAE and then liquid-solid separation; and the obtained liquid phase is subjected to concentration and alcohol precipitation to obtain a precipitate, namely, a soy hull polysaccharide.

In the present invention, the coarse material of soy hulls has a mass-volume ratio, preferably of 1 g:(15-25) mL, more preferably of 1 g:(18-22) mL and most preferably of 1 g:20 mL, with the extractant solution. In the present invention, the mass-volume ratio of the coarse material of soy hulls to the extractant solution is not limited to the g:mL level, and it can be scaled up or down in the same proportion, such as kg:L. In the present invention, the extractant solution has a mass fraction preferably of 0.5% to 0.7%, and more preferably of 0.6%. In the present invention, the extractant solution includes an ammonium oxalate solution. In the present invention, the MAE is conducted preferably at 83° C. to 88° C., and more preferably at 85° C.; the MAE is conducted with power preferably of 450 W to 500 W, and more preferably of 480 W; and the MAE is conducted preferably for 30 min to 40 min, and more preferably for 35 min. In the present invention, after the MAE, liquid-solid separation is conducted; and the liquid-solid separation preferably includes filtering and centrifuging conducted in sequence. In the present invention, the filtering is conducted preferably with double-layer gauze, and the filtrate obtained from the filtering is collected and centrifuged; the centrifuging is conducted preferably at 3,500 rpm to 4,500 rpm, and more preferably at 4,000 rpm; and the centrifuging is conducted preferably for 8 min to 12 min, and more preferably for 10 min. In the present invention, a liquid phase is obtained after the centrifuging, and the liquid phase is concentrated; and the concentrating is conducted until the liquid phase has a volume that is preferably 30% to 35% of the original volume, and more preferably ⅓ of the original volume. In the present invention, after the concentrating, the method further includes adjusting the pH of the concentrated solution preferably to 3.9 to 4.1, and more preferably to 4.0. In the present invention, the reagent for adjusting pH is preferably 1 mol of HCl. In the present invention, the alcohol precipitation preferably adopts absolute ethanol; the absolute ethanol is used at a volume that is preferably 2.8 to 3.2 times and more preferably 3 times the volume of the concentrated solution; and the alcohol precipitation is conducted preferably at 15° C. to 25° C., and more preferably at 20° C. In the present invention, after the alcohol precipitation, the method further includes standing treatment; the standing treatment is conducted preferably at 3° C. to 5° C., and more preferably at 4° C.; and the standing treatment is conducted preferably for 8 h to 12 h, and more preferably for 10 h. In the present invention, after the standing treatment, filtering and drying are preferably conducted to obtain a soy hull polysaccharide. In the present invention, the filtering is conducted preferably with gauze; the drying is preferably oven drying; and the oven drying is conducted preferably at 63° C. to 68° C., and more preferably at 65° C. The present invention has no special limitation on the time of the oven drying, provided that the material is dried to constant weight. The drying equipment is preferably a blast drying oven. In the present invention, the soy hull polysaccharide obtained by the above preparation method is subjected to structural identification, and it is determined that the soy hull polysaccharide has a structure shown in formula I.

The present invention also provides the use of a soy hull polysaccharide in the preparation of FSMPs. The present invention has no special limitation on the specific form and the raw materials and adjuvants of the FSMPs, and conventional raw materials and adjuvants for food in the art may be adopted. In the present invention, the soy hull polysaccharide, as a functional factor, is added to FSMPs at an amount preferably of 1% to 99%. In the present invention, the soy hull polysaccharide is derived from soybeans that are widely available, which can be consumed for a long time without toxic and side effects, and is hugely beneficial to human health.

The technical solutions provided by the present invention will be described in detail below with reference to examples, but the examples should not be construed as limiting the claimed scope of the present invention.

### EXAMPLE 1

Soy hulls were purchased from Shandong Yuwang Co., Ltd, and the soy hulls were prepared from Heihe 43 by the dry-peeling technology. The dry soy hulls were crushed and sieved by a 20-mesh sieve to remove protein residues and impurities. The resulting soy hulls were crushed once again by Twister, then sieved by a 60-mesh sieve, and decolorized. The resulting soy hulls were mixed with a 1% ethanol solution at a mass-volume ratio of 1 g:10 mL, and the resulting mixture was stirred at 20° C. for 30 min, and filtered with double-layer gauze. The residue was dried to constant weight at 65° C. in a blast drying oven. The dried coarse material of soy hulls was accurately weighed and then mixed with a 0.6% (w/v) ammonium oxalate solution at a mass-volume ratio of 1 g:20 mL. The reaction solution reacted at 85° C. under microwave (480 W, 60% of power) for 35 min, and then filtered with double-layer gauze. The filtrate was centrifuged at 4,000 rpm for 10 min, and then the supernatant was concentrated by rotary evaporation to have a volume ⅓ of the original volume. The pH of the concentrated solution was adjusted to 4.0 with 1 mol of HCl, and absolute ethanol, with a volume 3 times that of the concentrated solution, was slowly added at 20° C. under continuous stirring. The solution stood overnight at 4° C., then filtered with gauze, and dried at 65° C. in a blast drying oven to obtain a soy hull polysaccharide prepared by MAE.

### EXAMPLE 2

Influence of the Soy Hull Polysaccharide Obtained in Example 1 on the Dyslipidemia and Dysglycemia in Rats Induced by a High-Fat and High-Sugar Diet

Experimental Animals and Diets

Male SD rats, weighing 200±20 g, were purchased from the Laboratory Animal Center of Jinzhou Medical College. All animals were placed in an animal laboratory without specific pathogens under constant environmental conditions (12 h light-dark cycle; temperature: 24±1° C.; relative humidity: 60±10%), and had free access to food and water every day until the study ended. After one week of adaptation, 9 rats were fed with normal feedstuffs, and adopted as a normal control group. Another 9 rats were fed with high-fat and high-sugar feedstuffs (15.6% of protein, 31.1% of fat, and 53.3% of carbohydrate) for 4 weeks, and adopted as a model control group. Among the remaining 27 rats, 9 were fed with the soy hull polysaccharide at a high amount (800 mg/kg), 9 were fed with the soy hull polysaccharide at a medium amount (400 mg/kg), and 9 were fed with the soy hull polysaccharide at a low amount (100 mg/kg). During the experiment, samples of tail vein blood and intestinal feces were collected every week. Each treatment lasted 4 weeks, and all rats had free access to food and water.

All animal experiments were approved by the Animal Care and Use Committee of Jinzhou Medical University (Animal Experiment License No.: 2019015).

Growth Parameters

During the experiment, the body weight, daily water intake, and daily food intake of rats in each group were recorded at the 0, 1st, 2nd, 3rd, and 4th weeks, 5 times in total.

FBG

The FBG was tested 5 times for all rats at the 0, 1st, 2nd, 3rd, and 4th weeks. Blood samples were collected from the tail veins of fasting rats, and the FBG level was measured using a blood glucose meter.

Analysis of Serum Parameters

Blood samples were collected from the tail veins, and centrifuged (2,000 g, 1 min, 4° C.) to obtain serum samples. TG and HDL-C kits (Nanjing Jiancheng, China) were adopted, and the serum indexes were analyzed according to instructions of the manufacturer.

Extraction of DNA from Intestinal Bacteria, and 16S rDNA Gene Sequencing

Intestinal feces were collected from rats and stored at −80° C.

DNA was extracted from intestinal flora using QlAamp DNA Stool mini kit from QIAGEN. According to instructions of the kit, the intestinal feces were suspended in bacteriostatic EX buffer, and then DNA was bound to the QlAamp membrane for separation. The residual inhibitors and contaminants were removed by washing, and the complete DNA was eluted and purified from the QlAamp spin column.

The DNA was sent to Shanghai Biological Engineering Co., Ltd. for sequencing analysis.

Short-Chain Fatty Acids (SCFAs)

0.1250 g of each of acetic acid, propionic acid, i-butyric acid, butyric acid, i-valeric acid, and n-valeric acid standards was added to a 100 mL volumetric flask individually, and diethyl ether was added to obtain a stock solution. 1 mL, 0.75 mL, 0.5 mL and 0.25 mL of the stock solution were taken and added to 100 mL volumetric flasks separately, and diluted to 100 mL with diethyl ether. After the preparation was completed, the standards were analyzed, and a standard curve was plotted.

The dry feces samples were shaken thoroughly and then added with 2 mL of water (a phosphoric acid aqueous solution at 1:3), and the resulting mixture was vortexed and homogenized for 2 min. Extraction was conducted for 10 min with 2 mL of diethyl ether, and the resulting mixture was centrifuged at 4,000 rpm for 20 min (low temperature treatment, centrifuged under an ice water bath). After the centrifuging, the diethyl ether phase was separated, and for the remaining phase, extraction was conducted with 2 mL of diethyl ether at 4,000 rpm for 10 min. Finally, the obtained diethyl ether phase was separated and mixed with the previous diethyl ether phase, and the resulting mixture was subjected to volatilization to have a volume of 2 mL for injection analysis. The content of SCFAs was analyzed by the gas chromatograph-mass spectrometer (GC-MS). Chromatography conditions: TG WAX 30 m×0.25 mm×0.25 μm; carrier gas flow rate: 1.0 mL/min; inlet temperature: 240° C.; set temperature: maintaining at 100° C. for 5 min, increasing to 150° C. at 5° C./min, then increasing to 240° C. at 30° C./min, and maintaining at 240° C. for 30 min. Mass spectrometry (MS) conditions: interface temperature of GC-MS: 250° C.; ion source temperature: 200° C.; ionization mode: EI; and electron energy: 70 eV.

Induction of Type II Diabetes 5 weeks later, all rats were fasted for 12 h. Rats in the treatment group were intraperitoneally injected with streptozotocin (STZ) dissolved in 0.1 M citric acid/sodium citrate buffer (pH 4.5) twice at 40 mg/kg, and rats in the normal control group were intraperitoneally injected with the same amount of saline. After 3 days of feeding, a drop of blood was taken from the tail tip of the rat with a blood glucose meter to determine the blood glucose level for the rat. Rats that had an FBG concentration exceeding 11.1 mM suffered from induced type II diabetes.

Statistical Analysis

All data were expressed as mean±standard deviation (SD). SPSS 19.0 was used for statistical analysis. If the results of one-way analysis of variance (one-way ANOVA) show that p <0.05, the differences among experimental groups have statistical significance.

Results

Growth Parameters

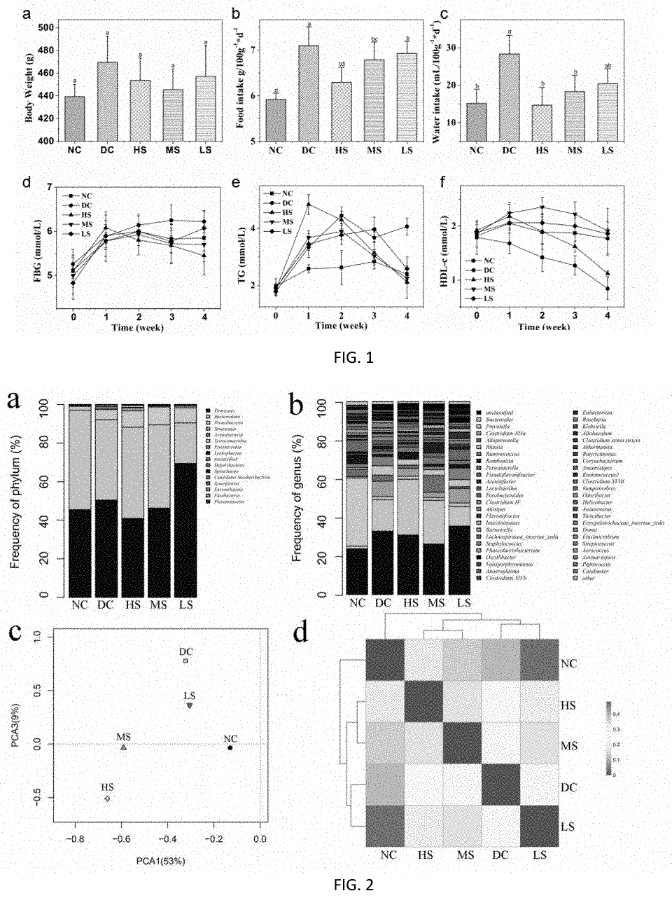

Rats with a high-fat and high-sugar diet were adopted as the research objects, and on the basis of intragastric administration of the soy hull polysaccharide, the role of the soy hull polysaccharide was studied in modulating the body weight gain, food intake and water intake. The changes of the body weight growth rate, water intake and food intake of each group were shown in a to c of FIG. 1. All rats gained weight during the obesity-inducing period. Compared with the model control group, the rats treated with the soy hull polysaccharide had decreased body weight. Compared with the normal control group, the group, administered with the soy hull polysaccharide at a medium amount, exhibited a lower body weight growth rate. In terms of food intake and water intake, the model control group is the highest; the normal control group is the lowest; the groups, intragastrically administered with the soy hull polysaccharide, are in an ascending order from the high-dosage group, to the medium-dosage group, and to the low-dosage group.

Biochemical Indexes

A high-fat and high-sucrose diet can cause high blood glucose and dyslipidemia, which is characterized by high level of total cholesterol and TG, and low level of HDL-C. Impaired FBG and dyslipidemia are common in diabetes and pre-diabetes. As shown in d of FIG. 1, the normal control group has a relatively-stable FBG level, while the model control group has an increased FBG level. Difference occurs between the normal control group and the model control group at the fourth week. The group administered with the soy hull polysaccharide has an FBG level lower than that of the model control group. Since the 2nd week, the FBG levels in groups, intragastrically administered with the soy hull polysaccharide at medium amount and high amounts, gradually decrease; and at the 4th week, compared with the normal control group, in groups intragastrically administered with the soy hull polysaccharide at high and medium amounts, the FBG levels decrease by 12.38% and 8.36% (p<0.05), respectively. These results may result from the viscosity and gelatinization of the soy hull polysaccharide, which can delay the diffusion of glucose and thus reduce the FBG level.

As shown by e and f in FIG. 1, compared with the normal control group, the group, intragastrically administered with the soy hull polysaccharide at a medium amount, has a significantly-raised serum TG content at the 1st week to the 3 rd week (p<0.05). However, at the 4th week, the group, administered with the soy hull polysaccharide at a medium amount, has a serum TG level lower than that of the normal control group. Compared with the model control group, the group, intragastrically administered with the soy hull polysaccharide at a medium amount, has a TG content that increases first, then decreases, and returns to the level of the normal control group at the 4th week. The results show that the group administered with the soy hull polysaccharide at a medium amount exhibits the best effect, followed by the group administered with the soy hull polysaccharide at a high amount. The administration of the soy hull polysaccharide at a medium amount can significantly increase the serum HDL-C content, and the group administered with the soy hull polysaccharide at a medium amount has an HDL-C level higher than the normal control group (p<0.05).

Modulation of the Structure of Intestinal Flora by the Soy Hull Polysaccharide

High-throughput sequencing (HTS) across the 16S rDNA V3-V4 hypervariable region (HVR) was adopted to determine the influence of the soy hull polysaccharide on the intestinal flora in rats with a high-fat and high-sucrose diet. In order to estimate the abundance and diversity of the intestinal microflora, Ace, Chaol, Shannon and Simpson indexes were calculated. As shown in Table 1, the group administered with the soy hull polysaccharide at a medium amount has the operational taxonomic unit (OTU), Ace index and Chaol index that are close to the normal control group. In addition, the group administered with the soy hull polysaccharide at a medium amount also has the Simpson index and Shannon index that are very close to the normal control group. These results indicate that the intake of soy hull polysaccharide makes the diversity of the intestinal flora reach a normal level.

TABLE 1
Influence of the soy hull polysaccharide on the diversity of intestinal flora in rats with a
high-fat and high-sugar diet

| | | | Index for abundance | | Index for diversity | |
|---|---|---|---|---|---|---|
| Groups | OTUs | Coverage | Ace | Chaol | Shannon | Simpson |
| NC | 881 | 1 | 1049.55 | 1035.19 | 4.27 | 0.06 |
| DC | 983 | 1 | 1284.08 | 1238.02 | 4.58 | 0.02 |
| HS | 1090 | 1 | 1359.33 | 1335.04 | 4.33 | 0.05 |
| MS | 945 | 1 | 1137.28 | 1106.64 | 4.27 | 0.04 |
| LS | 1189 | 1 | 1469.64 | 1391.64 | 4.88 | 0.02 |

The beneficial Firmicutes and Bacteroidetes are dominant in the human intestinal flora. As described above, it is found that the soy hull polysaccharide can significantly change the composition of the intestinal flora. Compared with the composition of the intestinal flora in rats of the model group, it is found that, in the group administered with the soy hull polysaccharide at a medium amount, at the phylum-level, the abundance of Bacteroidetes increases, and the abundances of Firmicutes and Actinobacteria decrease (a in FIG. 2). In order to determine the structural change of the intestinal flora, the relative abundances of the main floras in the four groups were compared (a to b in FIG. 2). There are significant differences among the groups in terms of the composition of the intestinal flora at all classification levels. In addition, at the genus-level, the treatment of the soy hull polysaccharide reduces the abundance of bacteria, including Blautia, Prevotella, Clostridium XlVa, Romboutsia, Barnesiella and Lachnospiracea incertae sedis. Compared with the model control group, Bacteroides, Alloprevotella, Pseudoflavonifractor, Flavonifractor, Phascolarctobacterium, Porphyromonas and Akkermansia are up-regulated, and the numbers of bacteria including Clostridium IV, Intestinimonas, Ruminococcus and Helicobacter return to normal levels (b in FIG. 2). The soy hull polysaccharide can provide a growth environment more beneficial for Bacteroidetes than for Firmicutes. A high-fat and high-sugar diet without dietary fiber will cause the intestinal flora imbalance, where the intestinal mucus is degraded, and the absorption of lipids and glucose is increased, resulting in the increase of FBG and TG levels, and the decrease of HDL-C level. After an appropriate amount of the soy hull polysaccharide is administered, the intestinal flora degrades the soy hull polysaccharide to maintain the balance of the flora, where the soy hull polysaccharide, in combination with the mucus barrier with high viscosity and hydrogel properties, prevents the rapid absorption of lipids and glucose, resulting in the decrease of FBG and TG levels, and the increase of HDL-C level.

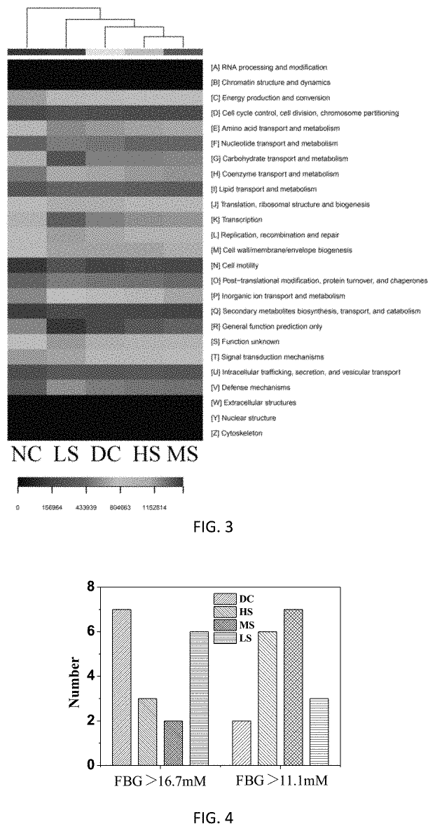

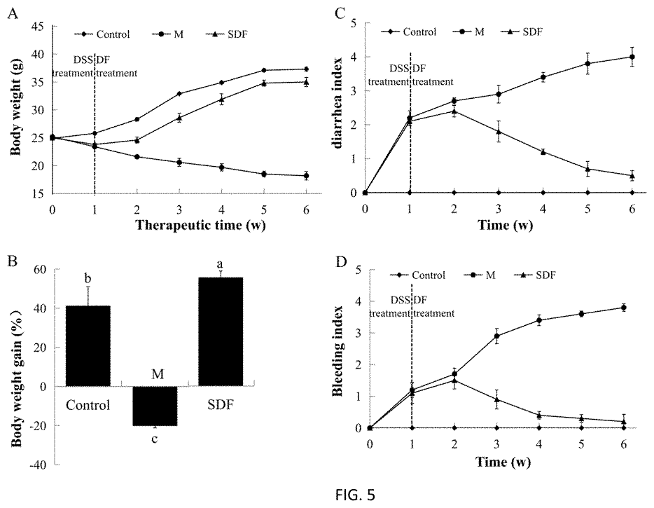

In order to evaluate the beta diversity, the PCA and the weighted principal coordinate analysis (PCoA) based on UniFrac distance were conducted for intestinal samples. The PCA score chart shows the similarity and variance among the normal control group, the model control group, the group administered with the soy hull polysaccharide at a high amount, the group administered with the soy hull polysaccharide at a medium amount, and the group administered with the soy hull polysaccharide at a low amount. The first three components explain 91% of the total variance (PC1, PC2 and PC3 account for 53%, 29% and 9%, respectively). The PCA was adopted to clearly classify the microorganism compositions of different treatment groups. As shown in c of FIG. 2, the differences among the five groups are statistically significant. The first two axes explain 62% of the total variation of the different groups. There is no overlap among these five groups of intestinal bacteria. In addition, the similarity among the samples can be intuitively seen from the coverage, which demonstrates that the carbon source has an influence on the intestinal microflora. In addition, the weighted PCoA based on weighted UniFrac (d in FIG. 2) illustrates how the soy hull polysaccharide changes the composition of the intestinal flora at the classification level. The PCA and weighted PCoA diagrams both show that the soy hull polysaccharide plays a role in modulating the structure of the intestinal flora; and further analysis confirms the influence of different carbon sources on the relative abundance and the composition of the intestinal flora. A heat map was plotted, where colors were used to reflect the abundance and function information, and the function and abundance were visually represented through the defined color depths (FIG. 3). The clustering heat map shows that rats in the model control group have a microbial profile different from that of rats in the normal control group. The treatment of the soy hull polysaccharide changes the microorganism composition. The composition is not similar to the normal control group, but is relatively-close to the normal control group in some aspects.

SCFAs

Due to changes in SCFAs and other metabolites, the intestinal microflora has a potential link with obesity, diabetes and other diseases. SCFAs, mainly acetate, propionate and butyrate, play an important role in the energy metabolism, modulation of lipogenesis, and glucose homeostasis. The contents of acetic acid, propionic acid, n-butyric acid, i-butyric acid, n-valeric acid and i-valeric acid are shown in Table 2. Except for the group administered with the soy hull polysaccharide at a low amount, the polysaccharide treatment groups have a total SCFA content significantly higher than that of the model control group (p<0.05). Rats intragastrically administered with the soy hull polysaccharide tend to recover. Obviously, acetic acid, propionic acid, n-valeric acid, and i-valeric acid are the main fermentation products of each model group, but the amount varies among the groups. The group administered with the soy hull polysaccharide at a medium amount has acetic acid, n-butyric acid, n-valeric acid and i-valeric acid contents that are significantly higher than that of normal rats (p<0.05). The group administered with the soy hull polysaccharide at a medium amount exhibits a significantly-improved yield of SCFAs. The results show that the group administered with the soy hull polysaccharide at a medium amount has the highest total SCFA content, with acetic acid, propionic acid and i-butyric acid as the main components. The group administered with the soy hull polysaccharide at a medium amount has a total SCFA content that is close to that of the normal control group, and is more than two times that of the model control group.

TABLE 2
Content of SCFAs

| | | Propionic | n-Butyric | | n-Valeric | | |
|---|---|---|---|---|---|---|---|
| Groups | Acetic acid | acid | acid | i-Butyric acid | acid | i-Valeric acid | Total |
| NC (μ/g) | 648.24 ± 3.27^{c} | 506.41 ± 0.86^{e} | 56.55 ± 0.69^{c} | 567.49 ± 1.22^{e} | 42.50 ± 0.66^{d} | 53.90 ± 0.09^{c} | 1875.09 ± 6.79^{a} |
| DC (μ/g) | 620.36 ± 0.76^{b} | 254.74 ± 0.15^{b} | 27.87 ± 0.48^{b} | 56.93 ± 0.57^{a} | 31.92 ± 0.52^{b} | 47.08 ± 0.14^{b} | 1038.9 ± 2.96^{d} |
| HS (μ/g) | 649.08 ± 2.19^{c} | 445.23 ± 0.36^{c} | 57.91 ± 0.61^{d} | 135.47 ± 0.88^{b} | 40.90 ± 0.83^{c} | 69.67 ± 0.16^{d} | 1398.26 ± 5.03^{c} |
| MS (μ/g) | 741.35 ± 1.99^{d} | 473.76 ± 0.84^{d} | 85.51 ± 0.58^{e} | 193.45 ± 0.26^{d} | 95.95 ± 0.87^{e} | 105.08 ± 1.09^{e} | 1695.0 ± 5.63^{b} |
| LS (μ/g) | 351.61 ± 1.10^{a} | 177.84 ± 0.24^{a} | 20.19 ± 0.17^{a} | 150.04 ± 1.24^{c} | 19.95 ± 0.06^{a} | 43.30 ± 0.33^{a} | 762.92 ± 2.8^{e} |

Note: Different letters in the same column indicate the significance of difference (p<0.05).

In rat models, the soy hull polysaccharide can reduce the occurrence of diabetes.

Three days after the injection of STZ, the blood glucose level was measured for rats. As shown in FIG. 3, it is found that rats in the groups administered with the soy hull polysaccharide have a blood glucose level significantly lower than that of the model control group (p<0.05); and the group administered with the soy hull polysaccharide at a medium amount exhibits the optimal effect. This indicates that the soy hull polysaccharide can inhibit the occurrence of diabetes. Long-term intake of the soy hull polysaccharide may change the structure and metabolites of the intestinal microflora, protect pancreatic β cells from damage, and prevent the insulin resistance.

### EXAMPLE 3

Alleviation of Inflammation in BALB/C Mice by Modulating the Intestinal Microflora With the Soy Hull Polysaccharide

Materials and Methods

The soy hull polysaccharide obtained in Example 1 is a polysaccharide of water-soluble dietary fiber (SDF), and the soy hull polysaccharide prepared in Example 1 is referred to as SDF hereinafter.

Functional Characteristics

Glucose Adsorption Capacity (GAC)

0.2 g of SDF was mixed with 20 mL of each of glucose solutions with different concentrations (50 mmol/L, 100 mmol/L and 200 mmol/L), and the resulting mixtures were incubated at 37° C. for 6 h. After the glucose adsorption reached equilibrium, the sample was centrifuged at 4,000 rpm for 15 min, and then the glucose concentration in the supernatant was determined with a glucose determination kit. GAC was calculated using the following formula: GAC (mmol/g)=Cᵢ−Cₛ×Vᵢ/Wₛ, where Cᵢ is the glucose concentration of the original solution (mmol/L), Cₛ is the glucose content in the supernatant when the adsorption reaches equilibrium; Wₛ is the weight of dietary fiber (g), and Vᵢ is the volume of the supernatant (mL).

Bile Acid Delay Index (BRI)

Sodium taurocholate was added to a 0.05 mol/L phosphate buffered saline (PBS) with a pH of 7 to obtain a sodium taurocholate solution with a concentration of 15 mmol/L. A dialysis membrane including 0.2 g of DF and a sodium taurocholate solution was placed in 100 mL of PBS (pH 7) at 37° C. (a sodium taurocholate solution without DF was adopted as a control). An aliquot part of dialysate (2 mL) was taken out for analysis at 1 h and 2 h, and the content of sodium taurocholate was measured by HPLC. The sodium taurocholate solution was injected into a Symmetry C18 (4.6 mm×250 mm) HPLC column with acetonitrile (reagent A) and disodium phosphate (0.15%, pH 3.0, reagent B) as mobile phases. All the above liquid phases were subjected to suction filtration. The linear gradient for the mobile phase was as follows: 22% to 42% A for 30 min, and then 42% to 35% A for 5 min, with a flow rate of 0.8 mL/min. A UV detector was used to detect taurocholic acid at 203 nm. BRI was calculated by the following formula: BRI (%)=100−[(C_{d}×100)/Cₑ], where, C_{d} and Cₑ are the total concentrations of taurocholic acids diffused out in the dietary fiber group and the control group (mmol/L), respectively.

Experimental Animals and Groups

Establishment of Mouse Models with DSS-Induced Colitis, and Treatment with SDF

Under conditions: no pathogen, 40% to 60% of humidity, and 26° C. to 28° C., 30 male BALB/C mice at 7 weeks old (18 g to 22 g) were raised in captivity under light-dark (12 h-12 h) cycles. The mice were randomly divided into a control group, a model group and an SDF group, with 10 animals for each group, and standard laboratory food and tap water were offered. The colitis models were established by induction with a 5% DSS solution for 7 days. The models were successfully established when the mice exhibited symptoms, such as diarrhea, bloody stool, body weight loss and laziness. 20 mice with colitis were intragastrically administered with SDF (100 mg/kg each time, 3 times a day). Only mice in the control group were not treated. The time and rate for establishing models were observed, including body weight, mental state, activity, and stool shape. All mice were evaluated weekly by body weight change, diarrhea index, and stool bleeding index. Blood and feces were collected before the mice were sacrificed. On day 42, the mice were anaesthetized with 3% diethyl ether, and then sacrificed by cervical dislocation. The scheme has been approved by the Ethics Committee of Gansu University of Chinese Medicine (No. 2019-206).

ELISA Analysis

A blood sample was collected from the heart, and then centrifuged (2,000 g, 1 min, 4° C.) to obtain serum. Then TNF-α was determined according to the ELISA kit protocol.

Western Blotting

Colon segments were collected from the control and treated animals, and then homogenized in ice-cold PBS and protease inhibitors. The total homogenate was subjected to lysis in RIPA lysis buffer with 1 mM PMSF for 1 h on ice, and then centrifuged at 15,000 g for 20 min at 4° C., and the supernatant was retained. The protein concentration was detected by the BCA kit. Proteins were separated by sodium dodecyl sulfate-polyacrylamide gel electrophoresis (SD S-PAGE) with 10% of running gel and 4% of stacking gel at 80 V for 2 h, then transferred to a membrane for 2 h. The membrane was blocked with PBS with 5% BSA for 1 h, and then incubated with the corresponding primary antibody of anti-TLR4, NF-κB and GAPDH at 4° C. overnight. Subsequently, the membrane was washed with PBS including Tween-20 for 30 min at room temperature, and then incubated with an HRP-labeled secondary antibody for 1 h at room temperature. The protein was washed with PBS including Tween-20 for 10 min at room temperature, and then detected using a chemiluminescence kit. The intensity of the protein was measured by AlphaView software.

HE Staining

At room temperature, a 1 cm colon segment was fixed in a 4% paraformaldehyde (PFA) solution for 12 h, and then embedded in paraffin wax. After the obtained paraffin was cut into 5 μm sections, the sections were dewaxed and finally washed with water, and then stained with 1% H&E at room temperature as described above.

The 16S rDNA gene sequencing has the same steps as the 16S rDNA gene sequencing in Example 2.

Statistical Analysis

Each experiment was conducted in triplicate. All data were expressed as mean±SD. SPSS 19.0 was used for statistical analysis. If the one-way ANOVA shows that p<0.05, the differences among experimental groups have statistical significance.

Results

Functional Characteristics

Table 3 lists the physical, chemical and functional characteristics of SDF. WRC and WSC of DF refer to the ability of DF to retain water in the matrix thereof. WRC of SDF is 7.95 g/g. WSC refers to the ratio of the volume of DF immersed in excess water after equilibrium to the actual weight of DF. WSC of SDF is 9.94 mL/g. OAC is used to evaluate the ability of DF to adsorb fat. OAC of SDF is 3.74 g/g. Fourier-transform infrared spectroscopy (FTIR) and X-ray diffraction analyses show that SDF has exposed hydrogen bonds and low crystallinity, thus indicating that SDF is easy to combine with water and oil.

It can be considered that GAC is an in-vitro index for the influence of DF on the delayed absorption of glucose in the gastrointestinal tract, which is helpful to assess the postprandial blood glucose level. Three glucose concentrations (50 mmol/L, 100 mmol/L, and 200 mmol/L) were adopted to evaluate the GAC of DF. GAC of SDF is 24.34% to 147.11%. BRI is used to predict the influence of DF on the delayed absorption of bile acids in the gastrointestinal tract. Taurocholic acid is obtained by esterification reaction of taurine and bile acid. Bile salts in the ileum or large intestine are metabolized and/or excreted into feces by fermentation in the colon. This process may be related to changes in the intestinal flora. The results show that BRI increases with the increase of dialysis time. BRI of SDF is 3.86% to 5.39%. High BRI means that more bile acids can be absorbed and the development of the intestinal mucosa can be prevented from being damaged.

TABLE 3
WRC, WSC, OAC, GAC and BRI values of SDF

| DF | WRC | WSC | OAC | GAC (mmol/g) | | | BRI (%) | |
|---|---|---|---|---|---|---|---|---|
| | (g/g) | (mL/g) | (g/g) | 50 mmol/L | 100 mmol/L | 200 mmol/L | 1 h | 2 h |
| SDF | 7.95 ± 0.11^{a} | 9.94 ± 0.78^{a} | 3.74 ± 0.02^{a} | 24.34 ± 0.21^{b} | 52.46 ± 0.15^{b} | 147.11 ± 0.10^{b} | 3.86 ± 0.33^{a} | 5.93 ± 0.29^{b} |

Influence of SDF on BALB/C Mice with DSS-Induced Colitis

As shown in A of FIG. 5, at the 1st week, the DSS-treated mice have a body weight significantly lower than that of the control mice. The SDF intervention also has a protective effect, and the body weight decreases. The number of mice with diarrhea and stool bleeding in the model group is significantly higher than that in the control group. However, DF intervention significantly reduces the number of mice with diarrhea and stool bleeding in each group (C and D in FIG. 5). Diarrhea and bleeding in the SDF group are significantly improved, which may result from the higher WRC, WSC and OAC of SDF. That may be because DF has strong hydrability, and can increase the stool weight, slow down the absorption of nutrients in the intestine, and alleviate the diarrhea and stool bleeding.

Inhibitory Effect of SDF on the TNF-α and NF-κB Pathways in the Colon Tissue

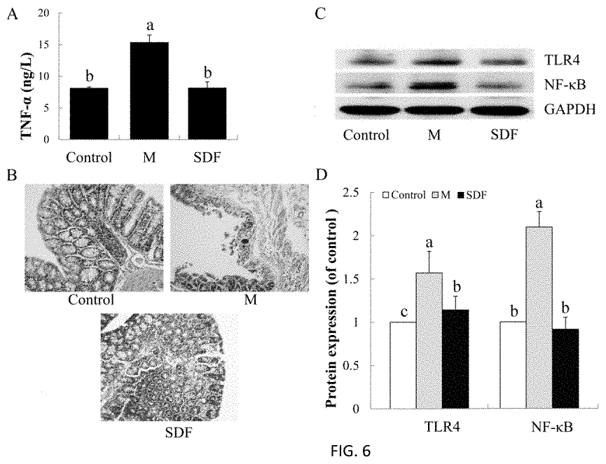

TNF-α is considered to be a key proinflammatory factor in the pathogenesis of a colitis model. In the DSS-induced colitis group, the serum TNF-a is released at an amount of 15.3 ng/L, nearly two times that of the normal control group (8.1 ng/L) (A in FIG. 6). After being administered with SDF, the mice with colitis exhibit a significantly-reduced increase in the release of TNF-α (p<0.05).

In order to determine the histological changes, H&E staining was conducted (B in FIG. 6). The normal control group exhibits a healthy colon tissue structure, with excellent shape, compact columnar epithelium, clear and separated layers between the mucosa and submucosa, intact intestinal crypts, and abundant goblet cells. In contrast, the colitis model group exhibits severe histological damage, irregular epithelial tissue, reduced goblet cells, and deformed crypt structure. Compared to the control group, in the colitis group, the gap between the mucosa and submucosa is greatly enlarged. However, in the SDF group, the colon tissue structure and the columnar epithelium structure are improved, goblet cells increase, and the gap between the mucosa and submucosa decreases.

Under pathological conditions, TLR is involved in the pathogenesis of many gastrointestinal diseases, including inflammatory bowel disease (IBD), colon cancer and infectious colitis. TLR-4 is activated by components in the bacterial cell wall, and this activation ultimately leads to the activation of the transcription factor of NF-κB. NF-κB induces inflammatory cytokines and growth factors, and is related to the pathogenesis of IBD. Therefore, inhibiting NF-κB is considered an important intervention measure for IBD. As shown in C and D of FIG. 6, the M and SDF groups have a content of TLR-4 protein significantly higher than that of the control group. The SDF group has a content of downstream NF-κB protein significantly lower than that of the M group. There is no significant difference between the SDF group and the control group. DF treatment can block the TLR-4/NF-κB inflammatory signaling pathway, which is related to the alleviation of colitis.

Influence of SDF on the Bacterial Diversity for Mice with DSS-Induced Colitis

The microflora in the colon contents was analyzed by HTS across the 16S rDNA V3-V4 HVR. Chaol and ACE values are positively correlated with the abundance of the intestinal flora. Shannon index is positively correlated with the diversity, while Simpson index is negatively correlated with the diversity (Table 4). Clean sequences of 68,468, 61,936 and 98,966 were obtained from the control group, M group and SDF group, respectively. Based on the similarity of 97%, OTU was adopted to cluster the clean sequences. The OTU, Shannon index and colony abundance (Chaol and ACE indexes, but excluding Simpson index) observed in all colitis model groups are lower than that of the control group. These results indicate that the colitis model groups have lower colony abundance and diversity. Under the treatment of DF, the OTU, Chaol index and ACE index are significantly higher than that of the colitis model groups (p<0.05), while the Shannon and Simpson indexes are close to normal. These results indicate that SDF improves the decrease in the abundance of intestinal flora caused by colitis.

TABLE 4
Sequencing data and alpha diversity for mice in each group

| Group | OUT | Shannon | Simpson | Chaol | ACE | Coverage |
|---|---|---|---|---|---|---|
| Control | 415 | 3.73 | 0.05 | 461.93 | 475.08 | 1.00 |
| M | 324 | 3.42 | 0.09 | 358.44 | 374.51 | 1.00 |
| SDF | 375 | 3.52 | 0.07 | 438.87 | 460.62 | 1.00 |

Modulation of the Structure of Intestinal Flora by DF

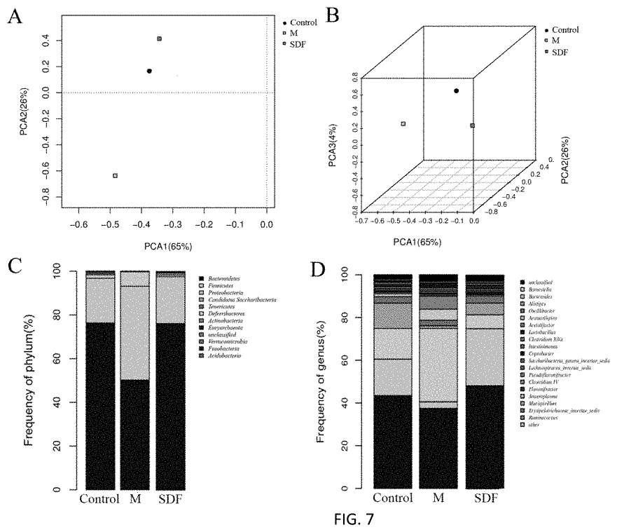

In order to evaluate the beta diversity, the PCA and the 3D-PCoA were conducted for intestinal samples (A and B in FIG. 7). The results of PCA and 3D-PCoA show that the control group and the DF group are clustered together, and are clearly separated from the colitis model group, indicating that the DF group and the colitis model group have significant difference in terms of the influence on the intestinal flora. In order to clarify the influence of different treatment methods on the composition of the microflora, the relative abundance (C and D in FIG. 7) and the proportions of the flora at the phylum-level and genus-level (Table 5) were determined. Firmicutes and Bacteroidetes are dominant in the human intestinal flora, and play an important role in modulation of absorption, energy conversion and glucose metabolism. As shown in Table 5, it can be intuitively seen from the numbers that the intestinal floras in mice are significantly divided into two categories, where the content of Bacteroidetes accounts for a large proportion. At the phylum-level, there are significant differences among the two intervention groups and the colitis model group (p<0.01; C in FIG. 7) in terms of the abundances of Bacteroides and Firmicutes. Compared with the normal control group, the colitis model group has a significantly-higher relative abundance for Firmicutes (p<0.01; C in FIG. 7). However, SDF treatment significantly reverses the increase induced by DSS (p<0.01), and increases the relative abundance of Bacteroides (p<0.001). The intestinal flora participates in the energy balance mechanism of the body. Firmicutes and Bacteroidetes play a key role in modulation of absorption, energy conversion and glucose metabolism. In addition, SDF treatment for DSS-induced mice also reduces the abundance of Proteobacteria, and increases the abundance of Actinobacteria at the phylum-level. At the genus-level, DSS significantly increases the relative abundance of Bacteroides and decreases the abundances of Alistipes and Barnesiella (p<0.01; D in FIG. 7). In the colitis model group treated with SDF, the relative abundances of Bacteroides, Alistipes and Barnesiella are significantly reduced (all p<0.01). Compared with the colitis model group, the DF group has higher relative abundances for Alistipes and Barnesiella, but a lower relative abundance for Bacteroides (p<0.05). In particular, the DF treatment group has a higher relative abundance for Barnesiella than the normal control group. Barnesiella degrades carbohydrates and regulates the immunity. In addition, compared with the colitis model group, the DF group exhibits higher abundances for Lactobacillus, Ruminococcus and Flavonifractor. Lactobacillus is a genus of bacteria that are beneficial to gastrointestinal health and can maintain the intestinal health by interacting with the immune system.

TABLE 5
Proportion of flora at phylum-level and genus-level (%)

| | Taxonomy | Control | M | SDF |
|---|---|---|---|---|
| Phylum | Bacteroidetes | 76.35 | 50.23 | 76.25 |
| | Firmicutes | 20.41 | 42.9 | 21.41 |
| | Proteobacteria | 1.62 | 6.62 | 0.8 |
| | Actinobacteria | 0.25 | 0.06 | 0.18 |
| Genus | Bacteroidetes \|Bacteroides | 14.37 | 34.32 | 6.54 |
| | Bacteroidetes \|Alistipes | 11.88 | 1.3 | 5.43 |
| | Bacteroidetes \|Barnesiella | 17.02 | 3.07 | 26.67 |
| | Firmicutes \|Lactobacillus | 0.92 | 0.13 | 0.98 |
| | Firmicutes \|Ruminococcus | 0.01 | 0.02 | 0.29 |
| | Firmicutes \|Flavonifractor | 0.07 | 0.71 | 0.46 |

It can be known from the above examples that the soy hull polysaccharide provided in the present invention can modulate intestinal flora, lower blood glucose and blood lipid, and prevent inflammation, and thus can be used in the preparation of drugs for modulating intestinal flora, lowering blood glucose and blood lipid, and preventing inflammation.

The above descriptions are merely preferred implementations of the present invention. It should be noted that a person of ordinary skill in the art may further make several improvements and modifications without departing from the principle of the present invention, but such improvements and modifications should be deemed as falling within the protection scope of the present invention.

1.-11. (canceled)

12. A method for modulating intestinal flora with drugs prepared from soy hull polysaccharide.

13. The method according to claim 12, wherein the modulating comprises promoting the proliferation of intestinal probiotics and increasing the diversity of intestinal flora.

14. A method for reducing blood glucose and blood lipid with drugs and foods for special medical purpose (FSMPs) prepared from soy hull polysaccharide.

15. A method for preventing and/or treating inflammation with drugs and FSMPs prepared from soy hull polysaccharide.

16. The method according to claim 15, wherein the inflammation comprises colitis.

17. The method according to claim 12, wherein, the soy hull polysaccharide is the only active ingredient in the drugs, and the soy hull polysaccharide has a mass percentage of 0.1% to 99% in the drugs.

18. The method according to claim 13, wherein, the soy hull polysaccharide is the only active ingredient in the drugs, and the soy hull polysaccharide has a mass percentage of 0.1% to 99% in the drugs.

19. The method according to claim 14, wherein, the soy hull polysaccharide is the only active ingredient in the drugs and FSMPs, and the soy hull polysaccharide has a mass percentage of 0.1% to 99% in the drugs and FSMPs.

20. The method according to claim 15, wherein, the soy hull polysaccharide is the only active ingredient in the drugs and FSMPs, and the soy hull polysaccharide has a mass percentage of 0.1% to 99% in the drugs and FSMPs.

21. The method according to claim 16, wherein, the soy hull polysaccharide is the only active ingredient in the drugs and FSMPs, and the soy hull polysaccharide has a mass percentage of 0.1% to 99% in the drugs and FSMPs.

22. The method according to claim 12, wherein a preparation method of the soy hull polysaccharide comprises the following steps:
1) crushing soy hulls and mixing the crushed soy hulls with an ethanol solution; stirring the resulting mixture at 15° C. to 25° C. for 25 min to 35 min; and then conducting filtering and drying to obtain a coarse material of soy hulls; wherein the ethanol solution has a volume fraction of 0.8% to 1.2%, and the soy hulls have a mass-volume ratio of 1 g:(8-12) mL with the ethanol solution; and
2) mixing the soybean seed coat coarse material with an extractant solution; conducting microwave-assisted extraction (MAE) and then liquid-solid separation for the resulting mixture; and subjecting the obtained liquid phase to concentration and alcohol precipitation to obtain a precipitate, namely, a soy hull polysaccharide;
wherein, the MAE is conducted at 83° C. to 88° C. for 30 min to 40 min, with power of 450 W to 500 W;
the soybean seed coat coarse material has a mass-volume ratio of 1 g:(15-25) mL with the extractant solution, and the extractant solution has a mass fraction of 0.5% to 0.7%; and
the extractant solution comprises an ammonium oxalate solution.

23. The method according to claim 13, wherein a preparation method of the soy hull polysaccharide comprises the following steps:
1) crushing soy hulls and mixing the crushed soy hulls with an ethanol solution; stirring the resulting mixture at 15° C. to 25° C. for 25 min to 35 min; and then conducting filtering and drying to obtain a coarse material of soy hulls; wherein the ethanol solution has a volume fraction of 0.8% to 1.2%, and the soy hulls have a mass-volume ratio of 1 g:(8-12) mL with the ethanol solution; and
2) mixing the soybean seed coat coarse material with an extractant solution; conducting microwave-assisted extraction (MAE) and then liquid-solid separation for the resulting mixture; and subjecting the obtained liquid phase to concentration and alcohol precipitation to obtain a precipitate, namely, a soy hull polysaccharide;
wherein, the MAE is conducted at 83° C. to 88° C. for 30 min to 40 min, with power of 450 W to 500 W;
the soybean seed coat coarse material has a mass-volume ratio of 1 g : (15-25) mL with the extractant solution, and the extractant solution has a mass fraction of 0.5% to 0.7%; and
the extractant solution comprises an ammonium oxalate solution.

24. The method according to claim 14, wherein a preparation method of the soy hull polysaccharide comprises the following steps:
1) crushing soy hulls and mixing the crushed soy hulls with an ethanol solution; stirring the resulting mixture at 15° C. to 25° C. for 25 min to 35 min; and then conducting filtering and drying to obtain a coarse material of soy hulls; wherein the ethanol solution has a volume fraction of 0.8% to 1.2%, and the soy hulls have a mass-volume ratio of 1 g:(8-12) mL with the ethanol solution; and
2) mixing the soybean seed coat coarse material with an extractant solution; conducting microwave-assisted extraction (MAE) and then liquid-solid separation for the resulting mixture; and subjecting the obtained liquid phase to concentration and alcohol precipitation to obtain a precipitate, namely, a soy hull polysaccharide;
wherein, the MAE is conducted at 83° C. to 88° C. for 30 min to 40 min, with power of 450 W to 500 W;
the soybean seed coat coarse material has a mass-volume ratio of 1 g:(15-25) mL with the extractant solution, and the extractant solution has a mass fraction of 0.5% to 0.7%; and
the extractant solution comprises an ammonium oxalate solution.

25. The method according to claim 15, wherein a preparation method of the soy hull polysaccharide comprises the following steps:
1) crushing soy hulls and mixing the crushed soy hulls with an ethanol solution; stirring the resulting mixture at 15° C. to 25° C. for 25 min to 35 min; and then conducting filtering and drying to obtain a coarse material of soy hulls; wherein the ethanol solution has a volume fraction of 0.8% to 1.2%, and the soy hulls have a mass-volume ratio of 1 g:(8-12) mL with the ethanol solution; and
2) mixing the soybean seed coat coarse material with an extractant solution; conducting microwave-assisted extraction (MAE) and then liquid-solid separation for the resulting mixture; and subjecting the obtained liquid phase to concentration and alcohol precipitation to obtain a precipitate, namely, a soy hull polysaccharide;
wherein, the MAE is conducted at 83° C. to 88° C. for 30 min to 40 min, with power of 450 W to 500 W;
the soybean seed coat coarse material has a mass-volume ratio of 1 g:(15-25) mL with the extractant solution, and the extractant solution has a mass fraction of 0.5% to 0.7%; and
the extractant solution comprises an ammonium oxalate solution.

26. The method according to claim 16, wherein a preparation method of the soy hull polysaccharide comprises the following steps:
1) crushing soy hulls and mixing the crushed soy hulls with an ethanol solution; stirring the resulting mixture at 15° C. to 25° C. for 25 min to 35 min; and then conducting filtering and drying to obtain a coarse material of soy hulls; wherein the ethanol solution has a volume fraction of 0.8% to 1.2%, and the soy hulls have a mass-volume ratio of 1 g:(8-12) mL with the ethanol solution; and
2) mixing the soybean seed coat coarse material with an extractant solution; conducting microwave-assisted extraction (MAE) and then liquid-solid separation for the resulting mixture; and subjecting the obtained liquid phase to concentration and alcohol precipitation to obtain a precipitate, namely, a soy hull polysaccharide;
wherein, the MAE is conducted at 83° C. to 88° C. for 30 min to 40 min, with power of 450 W to 500 W;
the soybean seed coat coarse material has a mass-volume ratio of 1 g:(15-25) mL with the extractant solution, and the extractant solution has a mass fraction of 0.5% to 0.7%; and
the extractant solution comprises an ammonium oxalate solution.

27. The method according to claim 22, wherein, in step 2), after the concentration, the preparation method further comprises adjusting the pH of the concentrated solution to 3.9 to 4.1; and absolute ethanol is used in the alcohol precipitation at a volume that is 2.8 to 3.2 times the volume of the concentrated solution.

28. The method according to claim 23, wherein, in step 2), after the concentration, the preparation method further comprises adjusting the pH of the concentrated solution to 3.9 to 4.1; and absolute ethanol is used in the alcohol precipitation at a volume that is 2.8 to 3.2 times the volume of the concentrated solution.

29. The method according to claim 24, wherein, in step 2), after the concentration, the preparation method further comprises adjusting the pH of the concentrated solution to 3.9 to 4.1; and absolute ethanol is used in the alcohol precipitation at a volume that is 2.8 to 3.2 times the volume of the concentrated solution.

30. The method according to claim 22, wherein the soy hull polysaccharide has a structure shown in formula I: [symbol]
wherein, ● is GalA, [symbol] is Ara, ▴ is Rha, and ▪ is Gal.

31. The method according to claim 27, wherein the soy hull polysaccharide has a structure shown in formula I: [symbol]
wherein, ● is GalA, [symbol] is Ara, ▴ is Rha, and ▪ is Gal.
